(19) United States
(12) Patent Application Publication
(10) Pub. No.: US 2016/0120187 A1
(43) Pub. Date: May 5, 2016

(54) Cartridge for foliar applications

(71) Applicant: George E. Hahn, Cardiff By The Sea, CA (US)

(72) Inventor: George E. Hahn, Cardiff By The Sea, CA (US)

(21) Appl. No.: 14/121,874

(22) Filed: Oct. 30, 2014

(51) Int. Cl. A01N 63/00; C12M 3/00; C12M 1/00

(52) U.S. Cl. CPC A01N 63/00; C12M 47/02; C12M 23/44

(57) ABSTRACT

The present invention describes a cartridge for its use in an apparatus for making a foliar liquid wherein the cartridge is prepackaged with a particulate matter consisting of beneficial microorganisms. The cartridge includes a seating means for retention of the particulate matter in an apparatus for aerating the particulate matter, and apertures permitting the passage of air through the cartridge.

Herein described is an apparatus utilizing aerating means for making a foliar liquid from particulate matter having beneficial micro-organisms for use as a foliar spray. More particularly, a cartridge for use in such an apparatus. This application is a divisional application of C-I-P application Ser. No. 12/930,77 filed Jan. 18, 2011, which was a continuation in part of Ser. No. 11/805,024, Filed: May 22, 2007.

### BACKGROUND

1. Field of the Invention

2. Description of the Related Art

The background for this invention is fully described in U.S. Pat. No. 6,475,503 B2, Methods of Using Worm Castings for Insect Repellency; U.S. patent applications Ser. No 10/247,054, Methods Using Worm Castings for Fungal Suppression; and U.S. patent application Ser. No. 10/299,350, Apparatus for Making a Foliar Liquid (the “'350 apparatus”), which application is incorporated herein by reference. Such patent and applications disclose a particulate means 70 (herein shown in FIG. 1) for receipt of a dry particulate matter used as an insect repellant or fungal suppressant, the particulate matter consisting of chitinase producing microorganisms isolated from worm castings wherein the enzyme count of the chitinase producing microorganisms is at least 1 million CFU/gdw (Colony Forming Units/gram dry weight), and an apparatus for making such insect repellant in foliar liquid. Each size of '350 apparatus has a specific volume and depth of water in which particulate matter is submersed, through which air is percolated by means of particulate means 70. This obtains the bubble mechanical force needed to fracture or separate the beneficial microorganisms from the particulate matter without destroying or smashing the microorganisms. However particulate means 70 has two main weaknesses in the operation of extracting the beneficial microorganisms:

(1) The particulate means 70 needs to hold the particulate matter at the bubble cavitation level to assure the highest energy to extract beneficial organisms from the particulate matter and to direct the bubbles up and through the particulate matter. The particulate means 70 has mesh on the sides as well as the bottom. The side mesh allows the aeration bubbles to bypass the particulate matter in the basket and evacuate the '350 apparatus without passing through the particulate matter. A closed side cartridge seals all of the air passageways except through the particulate matter.

(2) The correct particulate matter is very critical to proper balanced biological multiplication. An open basket invites the user to put together a particulate matter that does not have all of the balanced components. The closed cartridge comes with the correct particulate matter of components so a quality solution of foliar liquid is produced from each brew.

Cartridges of the prior art typically included of baskets of cylinder-shaped bodies having perforated tops, bottoms and side-walls wherein effluent water is percolated. This basket design permits particulate matter residue to escape to basket and be carried with the foliar liquid, as shown in U.S. Pat. No. 6,649,404 to Alms, et al. The cartridge of the invention solves the above limitations. The self contained cartridge can be pre-activated in situ so no particulate matter components are lost.

### BRIEF SUMMARY OF THE INVENTION

U.S. patent application Ser. No. 10/299,350 describes an apparatus for making a foliar spray. The invention described herein is a cartridge for accurately and consistently providing at least one particulate means for receiving a selected amount of particulate matter having beneficial micro-organisms.

### DRAWINGS

FIG. 1 is a perspective view of the particulate means of the prior art mounted in the cover of the liquid receptacle of the apparatus described in the '350 apparatus.

FIG. 2 is a perspective view, of a first embodiment of the cartridge of the invention.

FIG. 3 is a cross-section view of a first embodiment of the cartridge of the invention.

FIG. 4 is a perspective view of a first embodiment of the lid for the cartridge.

FIG. 5 is a perspective view of the cartridge showing the bottom of the canister.

FIG. 6 is a top view of a second preferred embodiment of the invention.

FIG. 7 is a front sectional view of the canister of the second preferred embodiment of the invention.

FIG. 8 is top view of a particulate containing pillow of the second preferred embodiment of the invention.

FIG. 9 is a side view of the particulate containing pillow of the second preferred embodiment of the invention.

FIG. 10 is the canister of FIG. 7 containing the pillow of the second preferred embodiment of the invention.

### DETAILED DESCRIPTION OF THE INVENTION

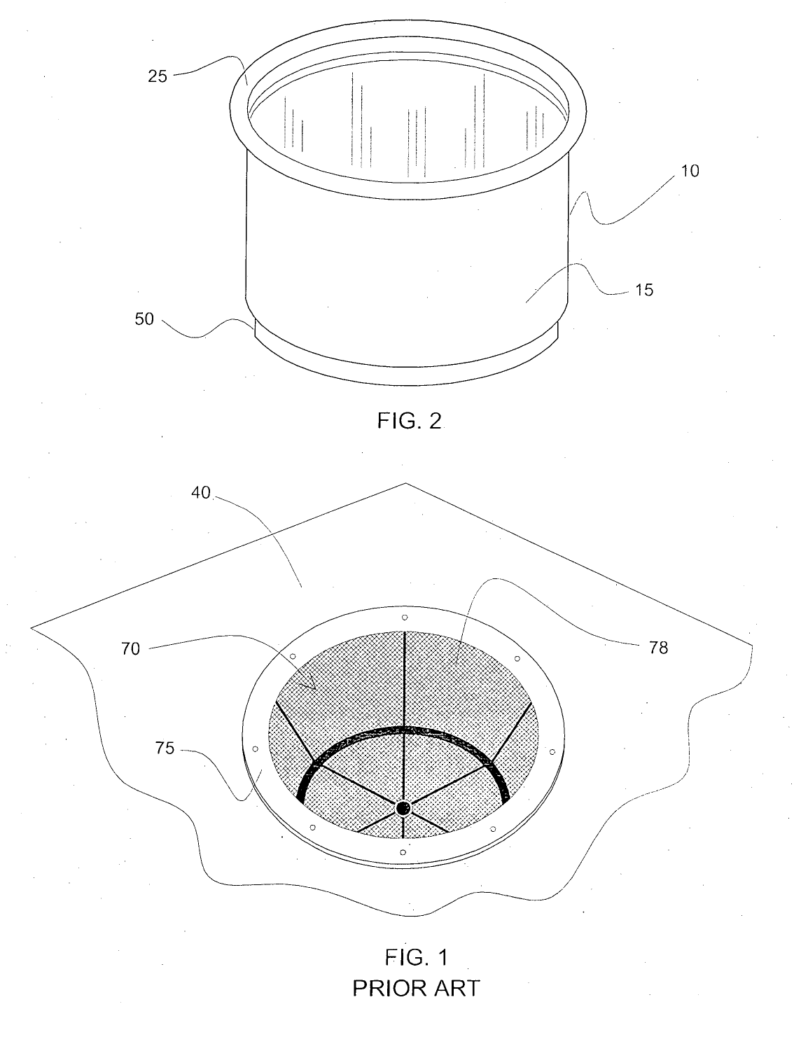

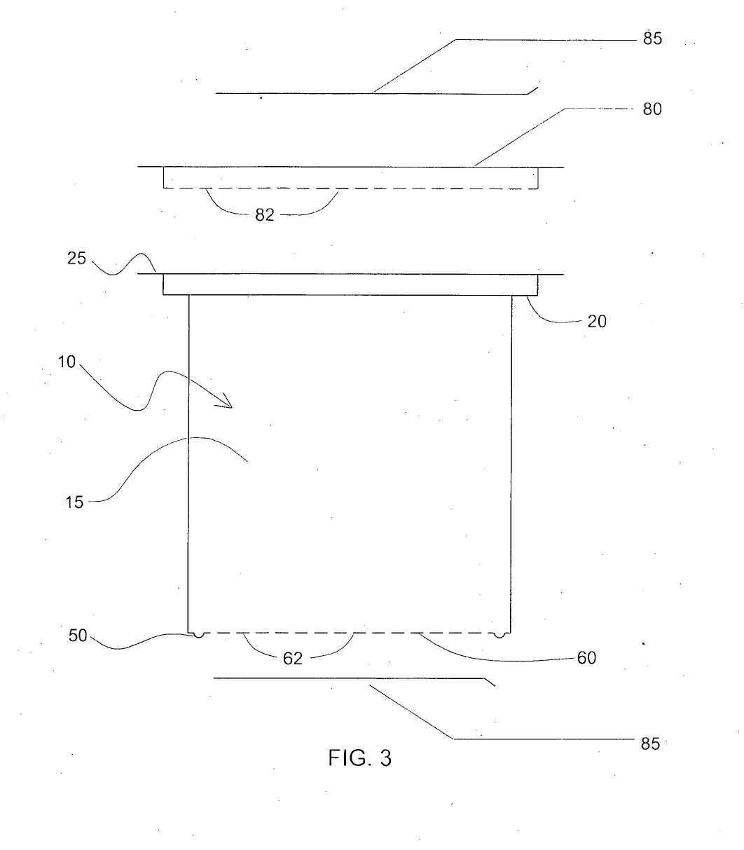

FIG. 1, describes a partial view of lid 40 of an apparatus (identified in FIG. 3 as lid 80 in the '350 apparatus) for making a foliar liquid described hereinabove. Particulate means 70 rests in a circular orifice of such lid. FIG. 2 describes the cylindrical, canister-like cartridge 10 of a first embodiment of the instant invention, which cartridge replaces particulate means 70 in such '350 apparatus. Cartridge 10 is sized to be snugly received in the orifice in the lid of the apparatus, and holds an appropriate amount of particulate matter so that when the aeration process of the '350 apparatus is complete, the concentration of beneficial organisms that has been fractured or separated from the particulate matter and suspended in the liquid is at a desired level. Cartridge 10 is the only means through which air injected by the aeration means of the '350 apparatus may be vented from the '350 apparatus during operation, thereby concentrating the bubble effect in the cartridge.

Referring to FIGS. 2 and 3, cartridge 10 is comprised of a canister 15 having an upper rim 20 and a bottom surface 50, and non-permeable sidewalls. Formed about the upper rim 20 is flange member 25. Flange member 25 is sized to abut against lid 40 of the '350 apparatus and maintain cartridge 10 in a fixed position in a holding ring installed in lid 40. Referring now to FIG. 5, canister 15 is shown to be formed with a recessed bottom, thereby creating a cylindrical rim 55. The purpose of this cylindrical rim 55 is to direct air from the aeration means of the '350 apparatus into the cartridge. Also shown in FIG. 5 are perforations 62 formed in bottom surface 60. Perforations 62 are sized to allow the passage of the equivalent air supplied by the aeration means of the '350 apparatus into cartridge 10. The surface area of perforations 62 is equal to the cross section of the volume of the airflow line increased by the compression ratio of the blower of the '350 apparatus. from cartridge 10. Perforations 62 are also sized to allow the bubbles (air) into the cartridge while holding the particulate matter (not shown) in the base of cartridge 10 until the particulate matter has been broken apart by the cavitation action of the breaking bubbles. The particulate matter includes, among other things, sand particles, cellulose fibers, compost, plant materials and beneficial organisms. Each size of '350 apparatus has a specific volume and depth of water. These figures determine the volume of air that is required to properly multiply the biology of the beneficial organisms in the particulate matter into a liquid form. The depth of cartridge 10 is sized to hold the body of cartridge 10 at the appropriate level in the '350 apparatus in order to absorb the maximum energy of the breaking bubbles from the aeration means. This is commonly referred to as “at the cavitation level.” The spacing of perforations 62 is selected to provide an even distribution of the air throughout the volume of cartridge 10.

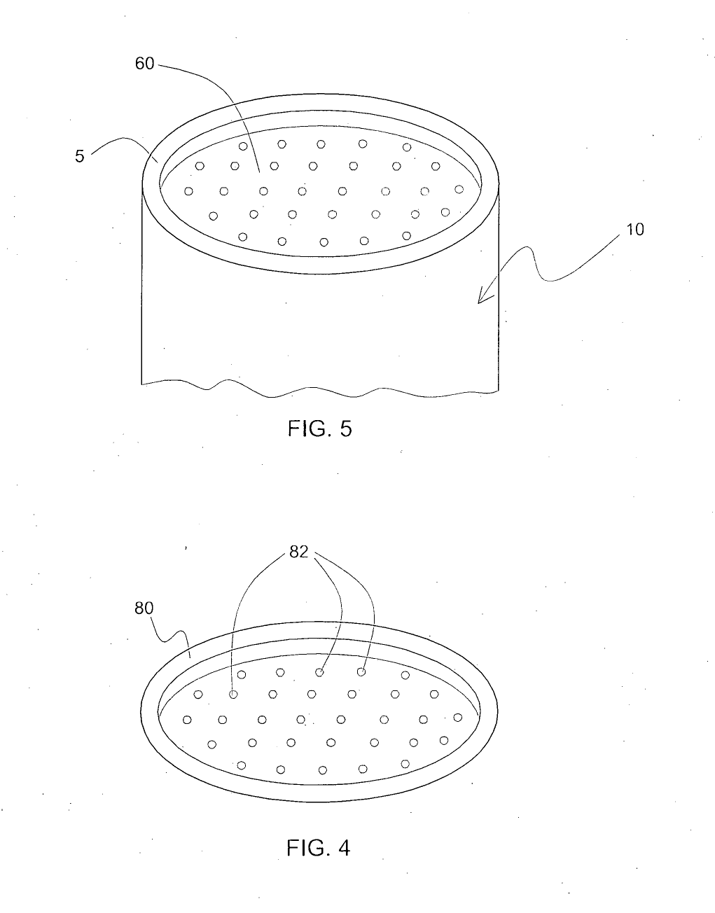

FIG. 4 discloses lid 80, which is formed to snugly and removably fit within the inside periphery of flange member 25. Also shown in FIG. 4 are perforations 82. Perforations 82 are sized to allow the passage of the equivalent air supplied by the aeration means of the '350 apparatus from cartridge 10. Lid 80 should fit sufficiently snug to restrict flow of air about its, perimeter, and direct the flow of air through perforations 82. In some cases, the perforations 82 are sized as two larger holes which have a secondary use as handling ports for the cartridge. Shown in the drawings but not critical to the invention are barriers 85, which are affixed to the top and bottom of the cartridge and which prevent leakage of particulate matter during transit and storage.

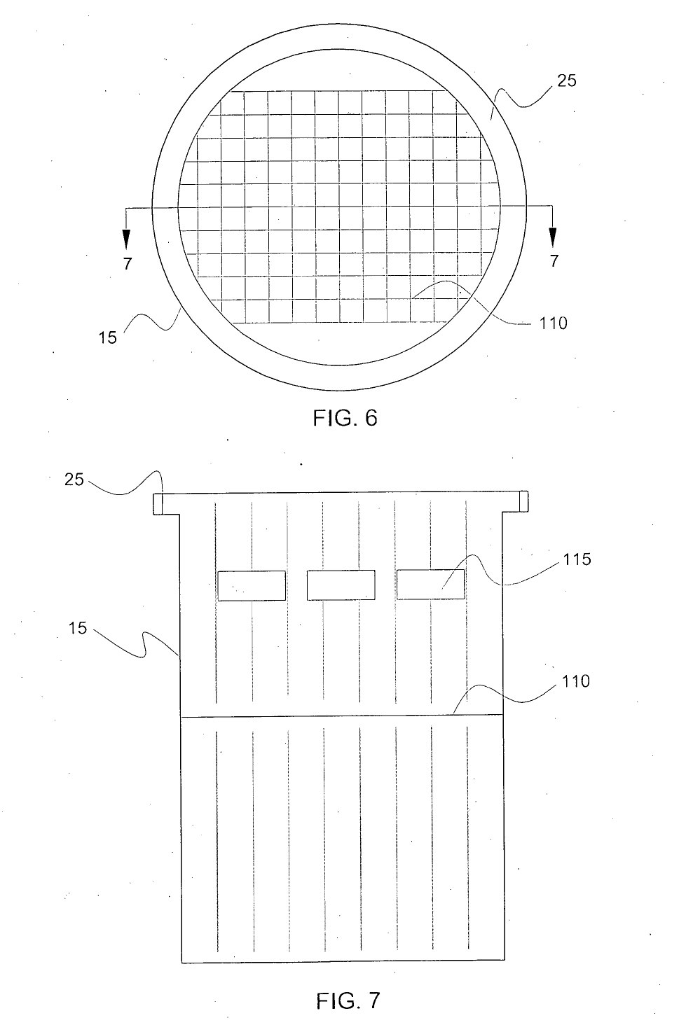

FIGS. 6-9 describe the second, preferred embodiment of the invention wherein the residue of the particulate matter is retained out of the foliar liquid after the beneficial organisms are separated out. In these figures like parts of the first embodiment are similarly numbered. FIG. 6 is a top view of canister 15 showing flange member 25 and grate element 110. Grate element 110 is constructed and sized to enable the maximum amount of aerated liquid to flow through canister 15 while supporting the particulate matter. FIG. 7 is a front sectional view of canister 15. In this embodiment the body of canister 15 is elongated, so as to funnel the aerated liquid through grate element 110. At some point intermediate between grate element 110 and flange member 25 are exit apertures 115, spaced about the periphery of canister 15, which permit the foliar liquid containing the beneficial organisms to flow out of canister 15. In this second preferred embodiment, perforations are not required in lid 80 (FIG. 4), so the only requirement is that lid 80 fit snugly in canister 15, such as by a screwing method.

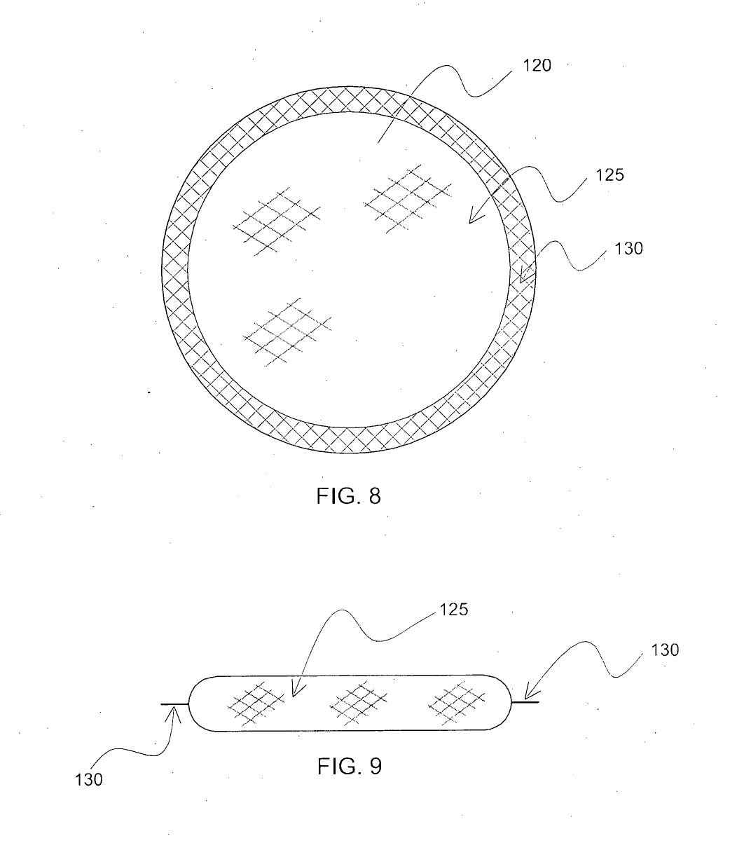

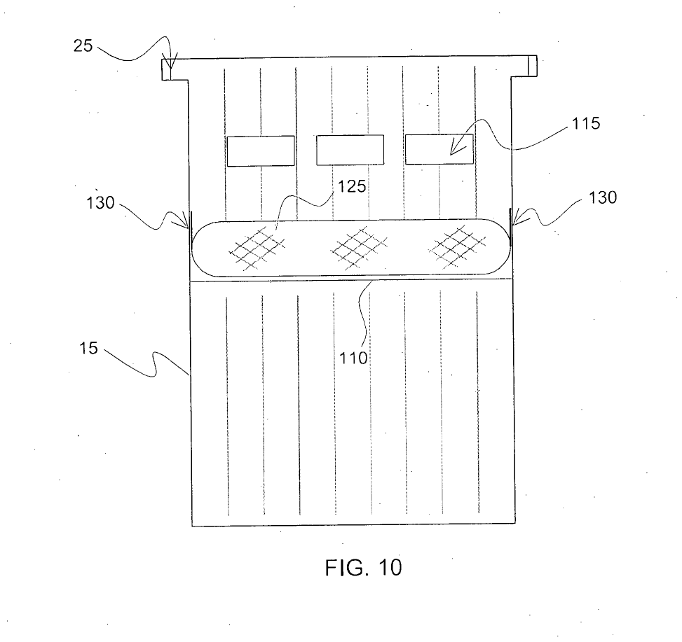

FIGS. 8 and 9 disclose a fabric pillow 120 for containing the particulate matter containing the beneficial organisms. FIG. 8 shows pillow 120 to have a circular profile, sized to nestingly fit within the body of canister 15 and rest on grate element 110. Pillow 120 consists of pillow body 125 and hem 130. Pillow 120 is constructed so that hem 130 which extends about the periphery of pillow body 125. When inserted in canister 15, as shown in FIG. 10, hem 130 serves as a gasket, or o-ring, to slideably seal the pillow 120 in canister 15 and to cause the aerated liquid to flow through pillow body 125 and not around the periphery of pillow body 125. As liquid is aerated and funneled through canister 15, the aerated liquid passes through pillow 120, fractures the beneficial organisms from the carrier particulate matter to be absorbed by the liquid to constitute the foliar liquid, and exits from canister 15 by means of apertures 115. Pillow 120 keeps the residue out of the foliar liquid by containing the non-beneficial particulate matter within the fabric pillow. Pillow 120 is fabricated from a synthetic polyethylene with a selected porosity to allow the beneficial organisms in the foliar liquid to pass through and the residue to remain inside the fabric. Although synthetic polyethylene is used in this preferred embodiment, the invention is not limited by the material used for pillow body 120. Any material that can withstand the aeration action of the liquid, have the requisite porosity to allow the foliar liquid to escape the pillow, and that will retain the non-beneficial particulate matter may be used.

The diameter of canister 15 is sized to snuggly fit in the lid portion of the '350 apparatus. It should be noted that there are various sizes of apparatus for making foliar liquids, and therefore, there may be various dimensions for canister 15. Concomitantly, there are various materials that may be selected for constructing canister 15. The only limitation is the corrosive elements or oxides not leach from the material during aeration of the foliar liquid. In these embodiments, plastic is the selected material.

In both embodiments of the invention, the particulate matter includes chitinase producing microorganisms isolated from worm castings wherein the enzyme count of the chitinase producing microorganisms is at least 1 million CFU/gdw (Colony Forming Units/gram dry weight).

While the present description contains many specificities, these should not be construed as limitations on the scope of the invention, but rather as an exemplification of some preferred embodiments thereof. Accordingly, the scope of the invention should not be determined by the specific embodiments illustrated herein, but rather in light of the full scope of the claims appended hereto.

I claim:

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. A cartridge for use in an apparatus for making a foliar liquid from a beneficial organism contained in particulate matter, the cartridge comprising:
(a) a removable canister for receipt of a particulate matter, the canister having an upper rim, the upper rim including a flange member around the periphery of the rim, the flange member for seating the cartridge in the apparatus for making a foliar liquid, and the cartridge and non-permeable cylindrical sidewalls;
(b) a grate for supporting the particulate matter, the grate positioned between the upper rim and a bottom periphery of the canister, the grate sized to permit the maximize flow of air and liquid through the grate;
(c) the non-permeable side walls having a plurality of apertures intermediate between the flange member and the grate, the plurality of apertures providing a flowpath for the foliar liquid from the canister;
(d) a lid for retaining the particulate matter in the canister; and
wherein the beneficial organisms are separated from the particulate matter and suspended in a foliar liquid when fractured by the passage of air through the canister.

15. The cartridge of claim 14 wherein the cartridge includes a permeable pillow for containing the beneficial organisms and particulate matter, the porosity of the pillow selected to permit air bubbles and foliar liquid through the permeable pillow while retaining non-beneficial particulate matter in the pillow.

16. The cartridge of claim 15 wherein the permeable pillow is circumferentially sized to nest within cylindrical sidewalls of the canister.

17. The cartridge of claim 1 wherein the particulate matter consists of chitinase producing microorganisms isolated from worm castings wherein the enzyme count of the chitinase producing microorganisms is at least 1 million CFU/gdw (Colony Forming Units/gram dry weight).
